(19) United States
(12) Patent Application Publication
(10) Pub. No.: US 2003/0034459 A1
(43) Pub. Date: Feb. 20, 2003

(54) Hygienic cabinet

(76) Inventor: Pete J. Bonin, Huntington Beach, CA (US)

Correspondence Address: GENE SCOTT, PATENT LAW & VENTURE GROUP ITTT, 3151 AIRWAY AVE, SUITE K 105, COSTA MESA, CA, 92626, US

(21) Appl. No.: 09/933,627

(22) Filed: Aug. 20, 2001

(51) Int. Cl. G01N 21/00

(52) U.S. Cl. 250/491.1

(57) ABSTRACT

A sanitization cabinet provides an air inlet passage mounted at a bottom panel. Air is drawn from the front of the cabinet and disbursed at the bottom interior of the cabinet. An interior surface provides a mirror finish and conformation for focusing light onto a shelf and surrounding area within the cabinet. An ultraviolet lamp is mounted within the cabinet in a position for sanitizing and heating the inlet air. An air outlet is positioned on top of the cabinet for exhausting the sanitizing air moving from bottom to top within the cabinet and which sanitizes articles within the cabinet. A timer circuit is activated when the door is opened and continues to energize the UV lamp for a set time after the door is closed.

### BACKGROUND OF THE INVENTION

1. Field of the Invention

This invention relates generally to equipment adapted for sterilizing and sanitizing of personal care object and medical implements, and more particularly to a cabinet for sanitizing common personal care objects such as a toothbrush or shaver blade.

2. Description of Related Art

The following art defines the present state of this field:

Smith, Jr., U.S. Des No. 281,476 describes a cabinet for toothbrushes, toothpaste or the like.

Eppler, U.S. Pat. No. 3,647,578 describes a receptacle for barber implements comprising: liquid-tight container means having a bottom, front wall, back wall, and end walls, lid means for covering the top of said container means, hinge means connecting said lid means with an upper edge of said back wall, generally rectangular tray means of a size to lie within and parallel to the bottom of said container, stop means protruding from the interior of said wall means, lever means pivotally mounted in said front and first position adjacent said bottom to a second position substantially parallel to but spaced from said bottom and adjacent said stop means, catch means for holding said lever means in said second position, and wherein said lid means is further provided with illumination means therein, and with switch means for activating said illumination means upon approach of said lid means to said container means.

Devon, U.S. Pat. No. 3,683,638 describes a drying and sterilizing cabinet especially suited for use in hospitals. The cabinet is sealed and has a dehumidifying chamber at its rear through which filtered air flows. Bacteria are killed by an arrangement of ultra-violet rays inside the cabinet.

Ozeki, U.S. Pat. No. 3,753,606 describes a film storing cabinet, comprising an outer casing with a door means and a film box swingably secured to the inside of the casing through a shaft means. The film box is selectively enclosed by the casing by closing the door means. Upon opening the door, the film box may swing about the shaft means so as to move its open end to the outside of the casing, and films or film-holding sheets slidably stored in the film box can selectively be brought out for inspection of films carried by the film-holding sheets.

Leittle, U.S. Pat. No. 3,776,694 describes a cabinet housing having a swingable door and an electric switch actuated by the door and an electric lamp disposed within the housing. The housing has openings for the emission of rays of light, and the lamp is actuated by opening and closing the door. A tooth brush rack is mounted in the housing, and another support member, for tooth water picks, combs, and the like, is also mounted in the housing for receiving these other items, all for sterilization of the tooth brushes and water picks and like items, by the action of the germicidal lamp, a unitary piece extends through the width of the cabinet at the top thereof and provides the rack, and a bracket is affixed to the unitary piece and supports the electric switch and shields the electric switch from being approached exteriorly.

Hayes, U.S. Pat. No. 3,807,824 describes a novel article of manufacture comprising a bathroom vanity cabinet having novel features of construction and possessing therefore particular advantages that make it attractive and useful to its owners or users. The problem of dealing with a small and possibly unsightly existing cabinet is dealt with by providing a larger structure having in a rear portion thereof an opening enabling it merely to be set in place over the existing cabinet. The new cabinet may be considerably larger, providing a larger mirror or mirrors, better lighting, more storage space for health and beauty aids, better electric-outlet availability, etc. The new cabinet has its own wiring system, with a plug that connects to the electrical system of the existing cabinet.

Moulthrop, U.S. Pat. No. 3,955,922 describes a sterilizer for bathroom articles for toothbrushes, dentures, combs, hairbrushes and the like comprises a box having a hinged lid with a mirror on the underside of the lid. A removable foraminous tray is supported above the bottom of the box and the items to be sterilized are placed thereon. A container of volatile disinfectant is disposed beneath the tray, as are also a fan and a plurality of ultra-violet lights. The mirror serves both as a vanity mirror and as a reflector to augment the action of the ultra-violet lights. The lights and the fan are in series so that the fan motor ballasts the lights.

Hall, U.S. Pat. No. 4,195,416 describes a cabinet adapted to be mounted on a wall of a washroom or similar facility with a conventional hand-held hair dryer secured within the cabinet and positioned so that the output of the hair dryer is projected through a vent opening in the cabinet. The hair dryer is controlled by a push-button time switch and the push button of the switch is operable from the outside of the cabinet. A suitable vaporizing substance is disposed within the cabinet adjacent to the inlet of the hair dryer so that the fumes are expelled through the vent together with the heated air. Additional electrical circuitry can be provided to override the time switch and to provide power outlets for washroom accessories such as electric shavers or lighting.

Smith, U.S. Pat. No. 4,314,733 describes a space saving filing cabinet designed to be deployed in a closet. The cabinet is an elongated narrow rectangular solid with a mounting tab on the top, which captures the hanger bar in the closet and stabilizer legs pivotally mounted to the bottom of the cabinet, which fan out and stabilize the cabinet when in use. Access is gained to the contents of the filing cabinet by withdrawing the inner cabinet from the outer cabinet. The inner cabinet slides forward on tracks mounted inside the outer cabinet. Once extracted the inner cabinet reveals drawers situated on the side face of the inner cabinet. Each drawer is a truncated rectangular box provided with a hinged door and two knobs. Each drawer is mounted within the inner cabinet by wheels, which follow grooves located in the inner cabinet, so that the bottom of each drawer will swing outwardly from the inner cabinet providing easy access to the contents of the drawer. An alternative mode of the present invention contemplates the use of the outer cabinet only. Access to the contents of the cabinet is gained through hinged doors provided on the outer face of the cabinet.

Cipelletti, U.S. Pat. No. 4,867,052 describes a device for maintaining in optimum hygienic condition the food mixture in the storage compartment and/or the delivery area of an espresso ice cream or whipped cream delivering machine. The storage compartment and/or the delivery area are treated by UV light or with ozone, preferably acting in an area closed by a door, which acts on the light or the supply of ozone.

Siegal, U.S. Pat. No. 5,160,699 describes an enclosed sanitizing cabinet for exposing eyewear articles to ultraviolet radiation from a fluorescent ultraviolet lamp, which preferably extends substantially the entire height of the cabinet. The cabinet contains shelf-forming rod members spaced and configured to support eyewear articles in such manner that the surfaces of said articles, which contact a user's skin in use, are exposed to ultraviolet radiation. Interior surfaces of the cabinet are of a nature and formed of materials so as to reflect ultraviolet radiation to all articles therein and to minimize the differential in radiation exposure between articles adjacent and more remote from the radiation source.

Zabsky et al., U.S. Pat. No. 5,185,532 describes a sterilizer is described that applies ultraviolet light to dental instruments such as slow and fast hand pieces, picks, etc., which applies the light largely uniformly to the forward portions of the instruments which are used in patients' mouths. The apparatus includes a plurality of supports (21-24) that are each constructed to hold a dental instrument, with the supports each rotatably mounted on a frame about a different vertical axis (31-34). An ultraviolet light source (18) directs light primarily horizontally at the forward portions of the dental instruments to sterilize them.

Walsten, U.S. Pat. No. 5,401,094 describes a recessed medicine cabinet using separate cabinet components related to a flush mounted cabinet and a recessed cabinet, each having a rear wall and peripheral walls projected forwardly therefrom and defining a storage space. The flush mounted cabinet component is larger both horizontally and vertically than the recessed cabinet component and a framed opening in a structural wall, suited to be mounted flush against the structural wall over the framed opening. The recessed cabinet component peripheral walls are sized to fit into the framed opening, and through an opening in the flush mounted cabinet component rear wall. The overall medicine cabinet storage space is comprised of both cabinet component storage spaces; the recessed cabinet component storage space lying rearwardly of the flush mounted cabinet component storage space. The flush mounted cabinet component can be positioned on the structural wall over the framed opening somewhat independently of its location, as the rear wall opening is located and formed on site as needed during cabinet installation to expose the framed opening for insertion of the recessed cabinet component into the framed opening.

Olsen, U.S. Pat. No. 5,577,819 describes a bathroom storage cabinet for electrically operated appliances such as hair dryers and hair curlers. Receptacles for the appliance cords are provided within the cabinet by a power bar, which is placed within an interior compartment utilized for neatly storing coils of electric cord excess length. A lower shelf and barrier wall retains the length of cord actually needed, so that a front door may be closed neatly flush with the cabinet, equipped with a magnetic latch arrangement. A lowermost compartment provides room for non-electrical brushes, combs and other bathroom paraphernalia and materials. The cabinet is adapted for installation embedded into a bathroom wall, or hanging from the face of the bathroom wall, or resting upon the bathroom counter.

Peifer, U.S. Pat. No. 6,096,264 describes a sterilizing cabinet for storing and sterilizing personal toilet articles, such as toothbrushes. The cabinet has a housing including a swinging door, receptacles for receiving and supporting a variety of differently configured electrically operated toothbrushes, and a rack having holes or slots for receiving and supporting manual toothbrushes. The cabinet has an electrical system having and supplying a sterilizing lamp capable of emitting ultraviolet radiation, an illuminating lamp capable of emitting visible light, and an AC-to-DC converter connected to recharging elements disposed within the receptacles for receiving electric toothbrushes. The sterilizing and illuminating lamps are independently switched. A safety switch senses door closure, and breaks power to the sterilizing lamp when the door is open. A charging receptacle connected to the AC-to-DC converter is mounted on the exterior of the cabinet.

The prior art teaches the use of ultraviolet energy sanitizing but does not teach a cabinet capable of using airflow and sanitizing methods for common item of personal hygiene. The present invention fulfills these needs and provides further related advantages as described in the following summary.

### SUMMARY OF THE INVENTION

The present invention teaches certain benefits in construction and use which give rise to the objectives described below.

A sanitization cabinet provides an air inlet passage mounted at a bottom panel. Air is drawn from the front of the cabinet and disbursed at the bottom interior of the cabinet. An interior surface provides a mirror finish and conformation for focusing light onto a shelf and surrounding area within the cabinet. An ultraviolet lamp is mounted within the cabinet in a position for sanitizing and heating the inlet air. An air outlet is positioned on top of the cabinet for exhausting the sanitizing air moving from bottom to top within the cabinet and which sanitizes articles within the cabinet. A timer circuit is activated when the door is opened and continues to energize the UV lamp for a set time after the door is closed.

A primary objective of the present invention is to provide an apparatus and method of use of such apparatus that provides advantages not taught by the prior art.

Another objective is to provide such an invention capable of sanitizing airflow for assuring a sanitary state for common items used in personal hygiene.

A further objective is to provide such an invention capable of providing a directed airflow without an air handling equipment.

A still further objective is to provide such an invention capable of automatic operation based on manual movement of a front access door.

Other features and advantages of the present invention will become apparent from the following more detailed description, taken in conjunction with the accompanying drawings, which illustrate, by way of example, the principles of the invention.

### BRIEF DESCRIPTION OF THE DRAWINGS

The accompanying drawings illustrate the present invention. In such drawings:

FIG. 1 is a perspective view of the invention showing an interior detail thereof;

FIG. 2 is a bottom perspective view thereof; and

FIG. 3 is an electrical circuit diagram thereof.

### DETAILED DESCRIPTION OF THE INVENTION

The above described drawing figures illustrate the invention in at least one of its preferred embodiments, which is further defined in detail in the following description.

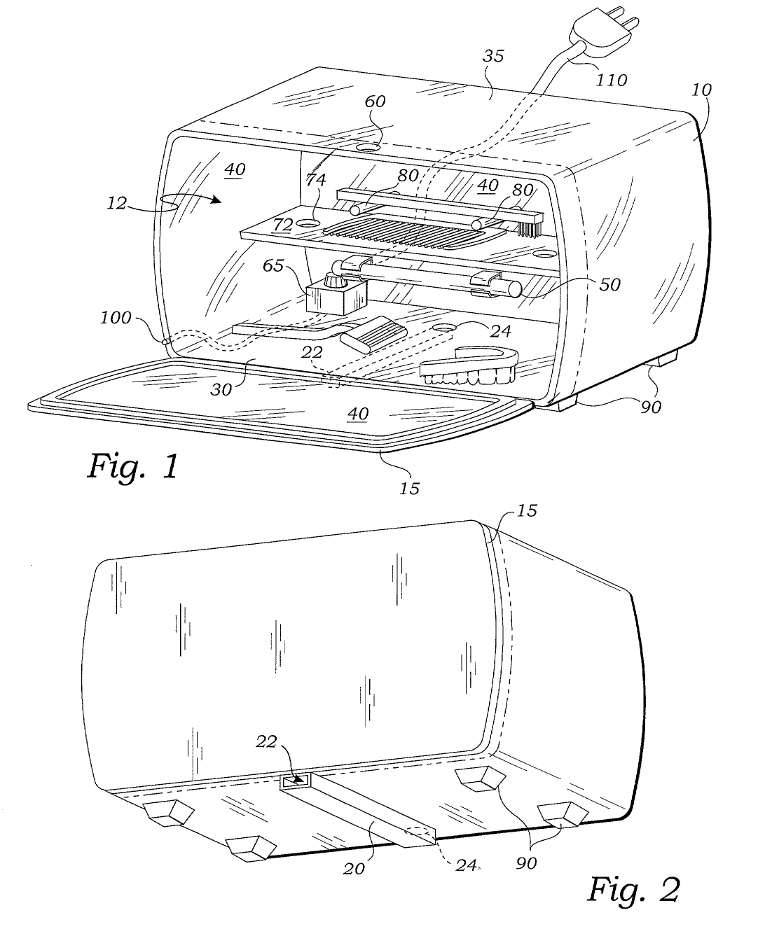

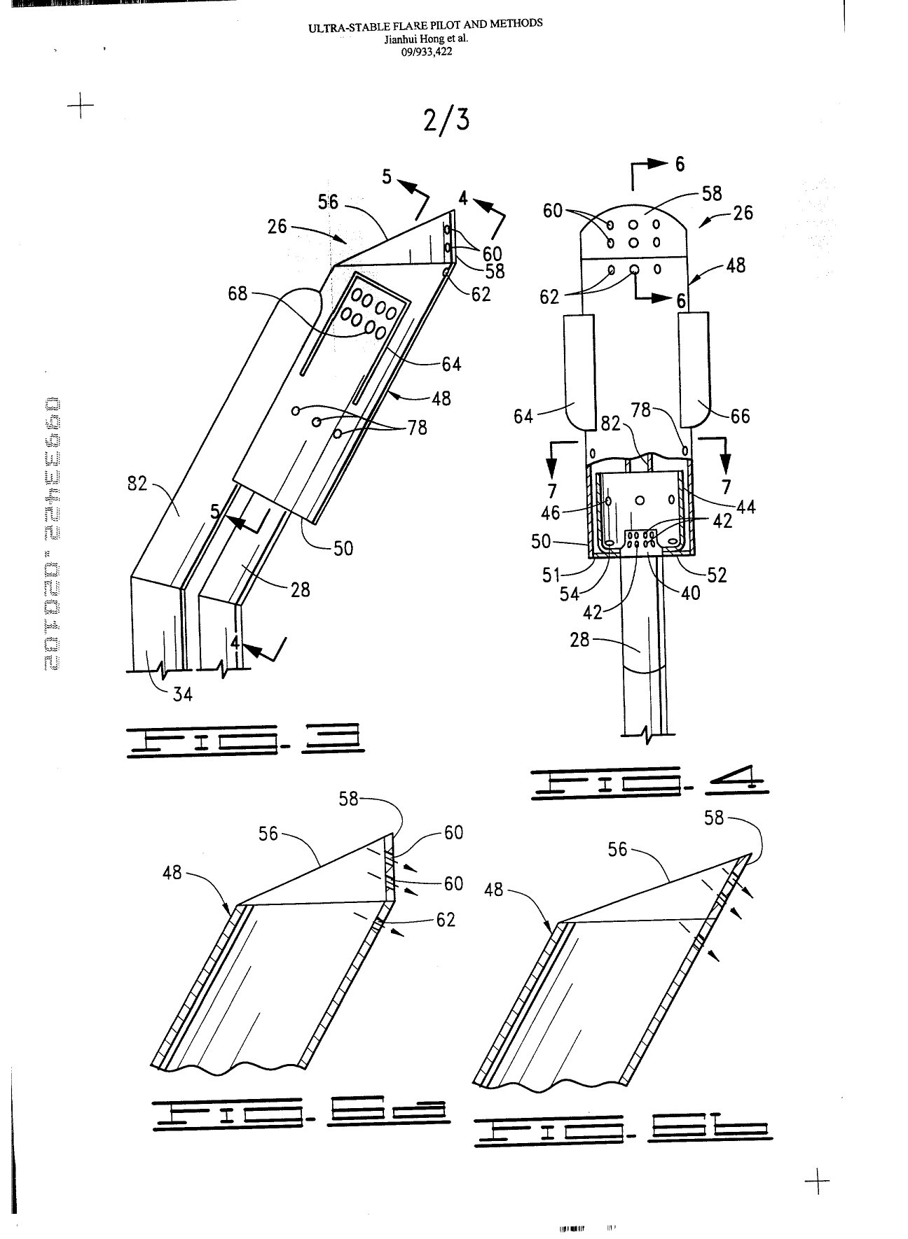

The present invention is a sanitization cabinet having an enclosure 10 with a movable door 15, preferably hinged downwardly as shown in FIG. 1, for ingress thereto at a front face 12 of the enclosure 10. The enclosure 10 provides an air inlet passage 20 mounted along a bottom panel 30 of the enclosure 10. This inlet passage 20 may be built into the panel 30 or may depend downwardly under the panel 30 as shown. The inlet passage 20 provides an external suction inlet aperture 22, at the front of the cabinet, and this aperture 22 is positioned adjacent to the front face 12 of the enclosure 10. The inlet passage 20 terminates with an internal suction outlet aperture 24 positioned within the bottom panel 30 so as to disburse air, drawn from the vicinity of the front of the cabinet into the interior of the enclosure 10. An enclosure interior surface 40 covers all interior walls of the enclosure 10. This interior surface 40 provides a mirror finish. An ultraviolet energy disbursing means 50, such as an ultraviolet lamp, is mounted within the enclosure 10 and an air outlet 60 is positioned in a top panel 35 of the enclosure 10. Air enters the enclosure 10 at the internal suction outlet aperture 24 after moving through the air inlet passage 20. It may be noticed from FIG. 1 that the air enters the enclosure 10 at the position of the ultraviolet energy disbursing means 50 and is heated by this energy. This heated air then rises within the enclosure 10 and exits at the air outlet 60. Thus a constant airflow is produced and this air is sanitized by the energy means 50.

A timer circuit 64 is adapted for driving the ultraviolet energy disbursing means 50 for a selected duration related to movement of the door 15. As can be seen in FIG. 3 power is supplied via AC power outlet cord 110. A timer 65 is adapted for manually setting a time duration to start when current flows in the circuit 64. Such timers are well known in the art. A door actuated switch 100 is wired for making the circuit 64 when the door is moved to the open position, shown in FIG. 1, at which time power is supplied to ultraviolet energy disbursing means 50. After the door is closed, again as sensed by the door switch 100, the timer 65 continues to enable current flow for a variable selectable time duration. Therefore, non-sanitized items placed within the enclosure, and any outside air entering the enclosure 10 when the door 15 is opened are sanitized when the door 15 is closed.

The enclosure interior surface 40 has at least one curved wall 42 adapted for concentrating reflections toward fixtures for holding items to be sanitized as will be described. A transparent shelf 70, preferably made of quartz, so as to be transparent to UV energy, is mounted within the enclosure 10 and positioned for receiving the air from the air inlet passage 20. The ultraviolet energy disbursing means 50 is positioned below the shelf 70. Therefore, the entering air is purified prior to moving into the general space areas of the enclosure 10. The shelf 70 provides a horizontal resting surface 72 for resting objects and articles to be sanitized, and this surface provides plural flow exhaust apertures 74 for allowing the sanitized air within the shelf to move into the general spaces of the enclosure 10. Plural resting pegs 80 are mounted on the interior surface 40 for supporting objects such as tooth brushes, razor blades and so on.

Plural feet 90 extend exteriorly downwardly from the bottom panel 30 of the enclosure 10 for resting the cabinet on any surface such as a vanity or table. The plural feet 90 are adapted by their length, for maintaining clearance between the resting surface and the air inlet passage 20. A phantom line shows the location of a wall when the apparatus is wall-mounted, i.e., only a small portion of the front of the apparatus protrudes from the wall. It is noted that the external suction inlet aperture 22 also protrudes from any mounting wall so that air is drawn from the room rather than from spaces within the wall in which the apparatus is mounted.

While the invention has been described with reference to at least one preferred embodiment, it is to be clearly understood by those skilled in the art that the invention is not limited thereto. Rather, the scope of the invention is to be interpreted only in conjunction with the appended claims.

What is claimed is:

1. A sanitization cabinet apparatus comprising: an enclosure having a movable door for ingress thereto at a front face thereof; the cabinet providing an air inlet passage mounted at a bottom panel thereof, with an external suction inlet aperture adjacent the front face of the cabinet, and an internal suction outlet aperture adjacent the bottom surface so as to disburse air into the enclosure; an enclosure interior surface covering all interiorly directed walls of the enclosure, the interior surface providing a mirror finish; and an ultraviolet energy disbursing means mounted within the enclosure and an air outlet positioned in a top surface of the enclosure.

2. The apparatus of claim 1 further comprising a timer circuit adapted for driving the ultraviolet energy disbursing means for a selected duration related to movement of the door.

3. The apparatus of claim 2 wherein the enclosure interior surface has at least one curved wall adapted for concentrating reflections therefrom.

4. The apparatus of claim 1 further comprising a quartz shelf mounted within the enclosure and positioned for receiving air exiting from the air inlet passage, the ultraviolet energy disbursing means positioned below the shelf.

5. The apparatus of claim 4 wherein the shelf provides a horizontal resting surface, the resting surface providing air flow exhaust apertures therein.

6. The apparatus of claim 4 wherein the interior enclosure provides plural resting pegs.

7. The apparatus of claim 1 further comprising plural feet extending downwardly from the bottom surface of the enclosure for resting the enclosure thereon, the plural feet adapted for raising the bottom surface such that the air inlet passage is spaced apart from a surface upon which the plural feet are in contact.
